(19) United States
(12) Patent Application Publication
(10) Pub. No.: US 2017/0096621 A1
(43) Pub. Date: Apr. 6, 2017

(54) A METHOD OF PREVENTING POLYMER TAR BUILD-UP IN ACH PRODUCTION OF MMA AND MAA

(71) Applicant: Lucite International UK Limited, Southampton, Hampshire (GB)

(72) Inventor: Neil Tindale, Cleveland (GB)

(21) Appl. No.: 15/126,784

(22) PCT Filed: Mar. 18, 2015
(86) PCT No.: PCT/GB2015/050794

§ 371 Date: Sep. 16, 2016

(30) Foreign Application Priority Data
Mar. 20, 2014 (GB) 1405006.6

(51) Int. Cl. C11D 1/83; C07C 51/06; C07C 231/06; C07C 67/20

(52) U.S. Cl. CPC C11D 1/83; C07C 67/20; C07C 51/06; C07C 231/06

(57) ABSTRACT

A method of preventing polymer tar build-up in ACH production of MAA and/or MMA is described. The method is characterised in that one or more surfactants are contacted with the hydrolysis and optional esterification stage reaction medium, the said surfactants are selected from: a) C₁₀ to C₃₀ alcohol ethoxylates with an average of 5 to 100 ethylene oxide units per molecule; b) alkyl, hydrogen, —O—[CH₂CH₂O]ₓH and/or —O—[CH₂CH₂CH₂O]ₓ H N-substituted alkylene di- or triamines with an average of 1 to 30 total ethylene oxide and propylene oxide repeating units per molecule and wherein x and x′ are from 1 to 30; and c) C₁₀ to C₃₀ alcohol ethoxylate, propoxylates with an average of 5 to 100 total propylene oxide and ethylene oxide units per molecule, which units may be in a random, block or alternating sequence or may be a combination thereof. The method is particularly useful for preventing build-up of oligomer and polymer tar-like deposits in reaction vessels, process equipment, pipework or other parts of the acetone cyanohydrin MMA and MAA production process.

The present invention relates to a method of preventing tar-like build up and blockages in methyl methacrylate (“MMA”) or methacrylic acid (“MAA”) production. Currently the most widely practiced process for the continuous production of methyl methacrylate (MMA) or methacrylic acid (MAA) is known as the “acetone cyanohydrin (ACH) route”. The invention relates to a method of preventing build-up of oligomer and polymer tar-like deposits in reaction vessels, process equipment, pipework or other parts of the acetone cyanohydrin MMA and MAA production process.

A number of commercial processes are used to prepare MMA. In one such process MMA is prepared from ACH. One embodiment of this process is described in U.S. Pat. No. 4,529,816. Generally, in the ACH process ACH is dissolved in, and hydrolysed by, an excess of concentrated sulphuric acid to produce in solution a mixture of Sulphatoisobutyramide (“SIBAM”) and Hydroxyisobutyramide (“HIBAM”). While still in the form of a solution in concentrated sulphuric acid, the HIBAM and SIBAM are thermally converted to methacrylamide (MAM) and a small amount of MAA. The ACH route to MMA or MAA is typically engineered as a continuous process, with output typically in the region of between 10 and 20 te/hr. The process steps from the initial mixing of ACH with concentrated sulphuric acid to the end of the thermal conversion of SIBAM and HIBAM to MAM may be collectively termed the “amide stage” of the process.

If the desired end product of the process is MAA, then the product of the amide stage of the process, being a concentrated sulphuric acid solution of MAM, is mixed with water, whereupon MAA is produced via hydrolysis of the MAM. If the desired product is MMA, the concentrated sulphuric acid solution of MAM is mixed with water and methanol, whereupon MMA is produced via a combination of hydrolysis and esterification of the MAM.

In order to facilitate the thermal conversion of SIBAM and HIBAM to MAM, both heat and residence time must generally be provided. A decrease in thermal conversion to the desired MAM results in a decreased overall yield for the process, and so high temperatures and relatively long residence times are typically used. Unfortunately, undesirable by-products are also formed in the amide stage of the process, and particularly in the high temperature thermal conversion stage. The undesirable by-products are made up of a wide range of chemical components, including many sulphonated compounds and also some oligomeric and polymeric materials.

The non-aqueous solvent properties of concentrated sulphuric acid are such that throughout the amide stage of the process, the undesirable by-products may remain dissolved in the reaction solution. However, when the reaction solution passes on into the hydrolysis or esterification process stages, water or water/methanol must be added to bring about the desired chemical conversion. The addition of water or water/methanol causes the properties of the solvent medium to change significantly, as a highly acidic aqueous medium is formed from a previously generally non-aqueous one. In this new solvent environment, any components which may have been soluble in the concentrated sulphuric acid but which are largely insoluble in the new medium will come out of the reaction solution, potentially forming small droplets or even solid particles in the solution. A process of agglomeration may take place so that larger droplets and particles and eventually deposits on the process reaction vessels, process equipment, pipework or other parts are formed.

The deposited material is typically referred to by those skilled in the art as “polymer tar” or just “tar”. The polymer tar is a highly viscous, sticky solid or liquid, and if untreated this will accumulate in process reaction vessels, process equipment, pipework and other parts. Blockage of such process parts in the hydrolysis or esterification stages of the acetone cyanohydrin process occurs when accumulation of a sufficiently large amount of deposit has taken place. The material is difficult to remove by conventional means such as pumping, chemical cleaning or dissolving.

The hydrolysis or esterification process steps of the ACH process generate MAA or MMA respectively, which may be recovered from the sulphuric acid reaction solution by processes such as liquid-liquid separation, distillation or steam stripping, to form a crude product which may then be subjected to further purification to produce a commercially pure product. After the recovery of crude MAA or MMA is complete, the remaining sulphuric acid containing mixture is known by those skilled in the art as “spent acid”, or “by-product acid”. Due to the relatively large volumes of spent acid produced from the acetone cyanohydrin route to MAA or MMA, and the relatively high cost of fresh sulphuric acid, the acid from the acetone cyanohydrin route to MAA or MMA is typically recycled in a separate process step known as a Sulphuric Acid Recovery (“SAR”) process.

Typical SAR processes are described in EP1057781 and U.S. Pat. No. 5,531,169, which both disclose SAR processes where the spent acid is introduced into a furnace in the form of aerosol droplets, along with fuel and air or oxygen. The fuel/air mixture is combusted to generate the necessary heat to vapourise, dissociate and decompose the acid along with any contaminants that may also be present to form mainly water, carbon dioxide, nitrogen and sulphur dioxide. The sulphur dioxide may then be recycled.

The aerosol droplets are typically produced in the SAR furnace by using a number of spray guns. The throughput of each of the spray guns is limited, and so a sufficient number of working spray guns must be provided to allow the processing of the complete volume of spent acid from the hydrolysis or esterification reaction to be managed.

Generally the spray guns work by forcing the liquid spent acid through a small diameter orifice under pressure. Unfortunately, the presence of polymer or other solid materials in the spent acid feed stream can block up the spray gun orifice, preventing further operation of the spray guns and thus a reduction in the production rate of MAA or MMA.

The problem of accumulation or polymer tars in the hydrolysis or esterification stage of the acetone cyanohydrin process such as in spray guns is typically managed by stopping the process, which is otherwise continuous, followed typically by draining, decontamination and cleaning by mechanical means. Such stoppages for clean-downs may for example, take between 2 and 4 days to accomplish, and in a continuous process, typically producing many tonnes per hour of product, any stoppage may represent a significant loss of earning potential. Clean-down stoppages are also undesirable because of the potential for exposure of those taking part in a clean-down activity to harmful sulphuric acid containing process liquid.

Operators of large scale continuous chemical plants are typically reluctant to add any new chemicals into their processes, because of the number of significant risks that this introduces which include:—

that a new chemical additive might take part in undesirable side-reactions with other components that are present;

that the new chemical additive might decompose in such a way that the product of the process may become contaminated with a new trace impurity;

that the reaction mix in which the new chemical is placed may start to foam; and

that the new chemical might cause corrosion or other damage to the process equipment.

Therefore, previous attempts at improving the task of polymer tar removal have been targeted at methods of dissolving of the polymer tar once the process has already been taken off-line and drained, for example, U.S. Pat. No. 6,245,216 discloses the use of strong acids and surfactants in combination with agitation to achieve a tar liquification effect.

Accordingly, for both economic and safety reasons, the avoidance of the formation and accumulation of significant deposits of polymer tars is highly desirable. Surprisingly, a method for treating the polymer tar within the acetone cyanohydrin route to MMA or MAA has been found.

According to a first aspect of the present invention there is provided a method of preventing polymer tar build-up in ACH production of MAA and/or MMA characterised in that one or more surfactants are contacted with the hydrolysis and optional esterification stage reaction medium, the said surfactants being selected from:—
a) C₁₀ to C₃₀ alcohol ethoxylates with an average of 5 to 100 ethylene oxide units per molecule;
b) alkyl, hydrogen, —O—[CH₂CH₂O]ₓH and/or —O—[CH₂CH₂CH₂O]_{x′}H N-substituted alkylene di- or triamines with an average of 1 to 30 total ethylene oxide and propylene oxide repeating units per molecule; and
c) C₁₀ to C₃₀ alcohol ethoxylate, propoxylates with an average of 5 to 100 total propylene oxide and ethylene oxide units per molecule, which units may be in a random, block or alternating sequence or may be a combination thereof.

By ACH production of MAA and MMA is meant the reaction of acetone cyanohydrin and sulphuric acid to eventually produce methacrylamide followed by hydrolysis and optionally esterification of the methacrylamide to methacrylic acid and methyl methacrylate. The hydrolysis and optional esterification stage reaction medium is that medium from which the MAA and/or MMA has been extracted or in which the MAA and/or MMA has been or is capable of being produced prior to extraction and generally includes sulphuric acid, tar/sludge, water and optionally, methanol.

The structure of the surfactants of the invention may be more particularly defined as follows:—

C₁₀ to C₃₀ alcohol ethoxylates with an average of 5 to 100 ethylene oxide units per molecule may be represented as formula I

R′″—O—[CH₂CH₂O]ₙ—H I

wherein R′″ is a C₁₀ to C₃₀ linear or branched alkyl group, preferably, a linear or branched C₁₀ to C₁₈ alkyl group, most preferably, a linear or branched C₁₃ to C₁₅ alkyl group and n is on average 5-100, preferably 5-30, more preferably, 6 to 20. Preferably, the R′″ group as defined and/or combined with the feature n above is branched such as a branched C₁₃ to C₁₅ alkyl group. Accordingly, a branched C₁₃ to C₁₅ alcohol ethoxylate with on average 6 to 20 such as 6 to 8 ethylene oxide units per molecule is particularly preferred.

The alkyl, hydrogen, —O—[CH₂CH₂O]ₓM and/or —O—[CH₂CH₂CH₂O]_{x′}H N-substituted alkylene di- or triamines may be represented by formula II

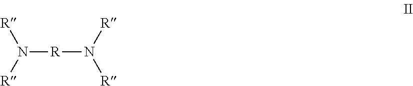

wherein

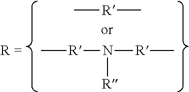

wherein R′ is a —C₂H₄— (ethylene) or C₃H₆— (propylene) group, each R″ is alkyl, hydrogen, —O—[CH₂CH₂O]ᵥH or —O—[CH₂CH₂CH₂O]ᵥH, wherein at least one R″ group, but not more than two R″ groups, is alkyl, preferably branched or linear C₁₀ to C₃₀ alkyl, more preferably branched or linear C₁₂ to C₂₅ alkyl, most preferably derived from one or more of stearic, palmitic, oleic, myristic, palmitoleic, linoleic and linolenic acid, especially tallow fatty acid and wherein at least one R″ group is —O—[CH₂CH₂O]ᵥH or —O—[CH₂CH₂CH₂O]_{v′}H, more preferably, at least one R″ group is —O—[CH₂CH₂O]_{V}H, and wherein the total average number of —[CH₂CH₂O]— and —[CH₂CH₂CH₂O]— repeating units per molecule of alcohol is from 1 to 20, preferably from 5 to 20, more preferably from 5 to 15 and wherein v and v′ are from 1 to 20 with the proviso that the total average number of such units does not exceed 20.

In certain preferred embodiments, the substituted alkylene di- or triamine includes or is of formula IIa

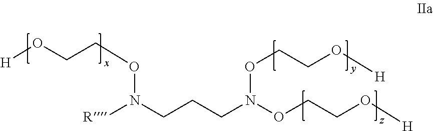

wherein R″″ is derived from tallow fatty acid such as tallow alkyl and x+y+z is on average 5-15 per molecule, preferably 8-12 per molecule.

The C₁₀ to C₃₀ alcohol ethoxylate, propoxylate defined in c) above may be represented by general formula III

R′″″—O—[CH₂CH₂O]ₙ—[CH₂CH₂CH₂O]ₘ—H III

where R′″″ is a linear or branched C₁₀ to C₃₀ alkyl group, preferably, a linear or branched C₁₀ to C₁₈ alkyl group, most preferably, a linear or branched C₁₃ to C₁₅ alkyl group, and n+m is on average 5-100 per molecule, preferably 20-50 per molecule, wherein the —CH₂CH₂O— and —CH₂CH₂CH₂O— units may be in a random, block or alternating sequence or may be a combination thereof.

In one preferred embodiment the surfactant (c) has an average of 20 to 50 total ethylene and propylene oxide units per alcohol and the alcohol group is a C₁₃ to C₁₅ alcohol represented as C₁₃ to C₁₅ alkoxy group.

Typically, in surfactant c), 1-99% of the units are propylene oxide units, more typically, 50-99%, most typically, 80-95%. Typically, in surfactant c), n:m is between 1:4 and 1:19, more preferably 1:8 to 1:10.

According to a further aspect of the present invention there is provided a method of producing methyl methacrylate (MMA) or methacrylic acid (MAA) comprising the steps of:—

converting acetone cyanohydrin to methacrylamide (MAM) using concentrated sulphuric acid and thermal treatment;

hydrolysing the MAM to MAA; and

optionally, esterifying MAA to MMA using methanol characterised in that one or more surfactants are contacted with the hydrolysis and optional esterification stage reaction medium, the said surfactants being selected from:—
a) C₁₀ to C₃₀ alcohol ethoxylates with an average of 5 to 100 ethylene oxide units per molecule;
b) alkyl, hydrogen, —O—[CH₂CH₂O]ₓH and/or —O—[CH₂CH₂CH₂O]_{x′}H N-substituted alkylene di- or triamines with an average of 1 to 30 total ethylene oxide and propylene oxide repeating units per molecule; and
c) C₁₀ to C₃₀ alcohol ethoxylate, propoxylates with an average of 5 to 100 total propylene oxide and ethylene oxide units per molecule, which units may be in a random, block or alternating sequence or may be a combination thereof.

Advantageously, use of the surfactants in the process of the present invention reduces blockages of process reaction process vessels, process equipment, pipework, spray guns or other parts. This may be achieved because the oligomer and polymer tar-like deposits surprisingly remain suspended in the acidic hydrolysing solution, in which condition they can be pumped away during subsequent stages of MMA/MAA purification or sulphuric acid recovery.

The present invention thereby provides for an increase in the periods between tar accumulation stoppages.

The surfactant is typically introduced into the reaction medium in such a way as to allow it to be well mixed in with other components. The process streams entering the hydrolysis or esterification stage of the process typically comprise a concentrated sulphuric acid solution containing MAM, streams of fresh water, fresh methanol (if used) and/or any recycle streams that may be returned to the process at this point, such as waste water or streams from the refining stages of the process.

Preferably, the surfactants are added to the process in liquid form either as substantially pure liquids at the temperature of addition, typically ambient temperature (25° C.), or in the form of a solution. This enables straightforward and accurate dosing of relatively small flows of surfactant by using for example metering pumps or flow meters.

The thorough mixing in of the surfactant within the hydrolysis or esterification medium vessel may be achieved by separate addition to the reaction medium at the same time as one or more of the other main components mentioned above, or a via a mixer placed in one of the other incoming process streams such as an in-line static mixer. A suitable incoming stream may be the fresh water and/or methanol stream or a recycle stream from the refining stage. As the liquids within the hydrolysis or esterification stage vessels are typically turbulent in nature, it is relatively facile to introduce all flows in such a way that they become well mixed in the reaction medium vessel in a relatively short period of time.

Of the surfactants of the invention, those defined in b) and c) together with their preferred features are preferred and surfactant b) together with its preferred features is more preferred.

By the hydrolysis and optional esterification stage reaction medium is meant the medium created by the addition of an effective amount of water or optionally water and methanol to the MAM containing concentrated sulphuric acid solution. For the avoidance of doubt the surfactants of the invention may be contacted with the medium either before or after MAA or MMA removal.

### EXAMPLES

In the case of the present invention, candidate surfactant types were screened using a series of lab scale tests. Descriptions of the tests used, and the type of results gained were as follows:

### Example 1: Miscibility with the Liquid Medium and Interaction with Polymer Tar

A straightforward visual observation test was used, in which a 100 ml glass beaker containing spent acid at 120° C. was placed upon the stage of a low powered microscope, and observations made when a 5% solution of polymer tar in concentrated acid was added first to spent acid as a control experiment, and then to spent acid containing a small amount of surfactant. In the control experiment, and those experiments where the candidate materials were clearly immiscible with the spent acid medium, or showed no positive interaction with the polymer tar, the tar would quickly agglomerate forming large particles, or stick to the side of the glass beaker. In those experiments where the candidate surfactants showed a positive interaction with the polymer tar, the tar would remain well dispersed in the spent acid in the form of small droplets, and would not stick to the walls of the glass beaker. A very large number of materials were subjected to this test. Some illustrative examples are shown in the table 1, below, in which it can be seen that only three candidate materials were found which were miscible with the hot BPA liquid medium, and also showed a sufficiently positive interaction with polymer tar to be included in subsequent tests.

TABLE 1
Initial Screening of Candidate Materials

| | Miscibility with | Interaction with |
|---|---|---|
| | BPA Medium | Polymer Tar |
| Candidate Material | (Yes or No) | (Yes or No) |
| Alkyl substituted | Y | N |
| polyethylenediamines | | |
| Tallow substituted | Y | N |
| ethylene diamine | | |
| Coconut oil | N | N |
| Palm Kernel oil | N | N |
| Nonylphenol ethoxylate, | Y | N |
| average (av) 25 ethylene oxide | | |
| (EO) units per alcohol | | |
| Mixed alkylbenzenes, | N | N |
| b.p. >150° C., (an industrial | | |
| solvent) | | |
| Alkyl-ether-carboxylic | N | N |
| acid | | |
| C7 alkyl substituted | Y | N |
| phenylenediamine | | |
| polyethylene | Y | N |
| oxide/polypropylene block | | |
| copolymer | | |
| Alkylphosphate ester | N | N |
| Alkylethoxylatephosphate | N | N |
| esters | | |
| Branched C13-15 alkyl | Y | Y |
| ethoxylate (av 7 EO units per | | |
| alcohol) | | |
| ‘N’ - ethoxylated, ‘N’ - | Y | Y |
| tallow substituted propylene | | |
| diamine (av 10 EO units per | | |
| molecule | | |
| Branched C13-15 alkyl | Y | Y |
| ethoxylate/propoxylate (av 20 | | |
| to 30 PO/EO units per | | |
| alcohol, of which 90% are PO) | | |

### Example 2: Foaming of Spent Acid

A purpose designed foam measurement device was used, which comprised a graduated glass tube with a sintered glass sparging device mounted within it such that the glass tube could be filled with liquid, and compressed air sparged in below the surface of the liquid. In the control case of spent acid with a few drops of polymer tar added, the flow of sparging air was adjusted until it caused a fixed height of foam to form above the liquid in the tube. To achieve the desired assessment, one drop of a candidate surfactant was then added to a foaming control experiment, and once the foam height had changed the new foam height was noted. Candidates which increased the height of the foam in the system were rejected. Results for the three successful candidate materials from Test 1 are shown in Table 2, below, in which it can be seen that all three candidates actually behaved as antifoam agents in the hot spent acid system.

TABLE 2
Results of Foaming Experiments

| | Increase or Decrease of foam height |
|---|---|
| Candidate Material | on addition to spent acid medium |
| Branched C13-15 alkyl ethoxylate | Decrease |
| (av 7 EO units per mol) | |
| ‘N’ - ethoxylated, ‘N’ - tallow | Decrease |
| substituted propylene diamine | |
| (av 10 EO units per molecule) | |
| Branched C13-15 alkyl | Decrease |
| ethoxylate/propoxylate | |
| (av 20 to 30 PO/EO units per | |
| alcohol, of which 90% are PO) | |

### Example 3: Corrosion

Coupons of the materials of construction of the vessels and equipment used in the hydrolysis and esterification stages of the process were suspended in stirred spent acid, with added polymer tar, at process temperature, at lab scale in glass equipment. To achieve the desired assessment of the surfactants, the candidates were each added to spent acid in separate experiments at a level of 1% w/w. After 1 week of stirring at process temperature the coupons were removed, rinsed and examined microscopically for any signs of corrosion damage. The results are shown in Table 3, below, in which it can be seen that none of the candidate materials caused an observable effect on corrosion of the coupons.

TABLE 3
Corrosion Experiments

| | Observations upon microscopic |
|---|---|
| | examination of coupons after exposure |
| Candidate Material | to spent acid |
| Control experiment, no | Slight very slight surface roughening |
| surfactant present | present, some black staining visible |
| Branched C13 alkyl ethoxylate | As control |
| (av 7 EO units per alcohol) | |
| ‘N’ - ethoxylated, ‘N’ - tallow | As control |
| substituted propylene diamine | |
| (av 10 EO units per molecule) | |
| Branched C13 alkyl | As control |
| ethoxylate/propoxylate | |
| (av 20 to 30 PO/EO units per | |
| alcohol, of which 90% PO) | |

### Example 4: Decomposition

The composition of the process liquid in the hydrolysis and esterification stages of the acetone cyanohydrin route process to MAA and MMA are understood to be highly acidic and corrosive. As such it is important that any materials that will come into contact with process liquid are stable against rapid, acid promoted chemical decomposition. To test the candidate surfactant materials, the beaker test described in Test 1 was repeated, but the duration of the test was extended. Any candidate showing only a temporary interaction with the polymer tar was rejected, as this was taken to be a sign that the material had decomposed in the hot, corrosive medium. The results of this testing on the three remaining candidates showed that all three had retained their effectiveness at dispersing the polymer tar after 30 minutes in the hot, spent acid medium.

### Example 5: Product Quality

MAA and MMA are both traded as essentially pure products, with purity specifications of greater than 99.9%. It is therefore important that trace impurities are kept to a very low level in the pure commercial products. It follows that the presence of any new close boiling impurities, which may have arisen as a result of impurities in a new additive, or from the decomposition of a new additive that was being used in the process, would be highly undesirable. For this reason when a new additive is proposed for use in the acetone cyanohydrin route to MAA or MMA process, it is important that a test can be carried out that clearly shows that no new trace impurities are found in the product that can be ascribed to the use of the new additive. With this requirement in mind candidate surfactant materials were subjected to model esterification reactions, at laboratory scale. The candidate surfactants were each added to separate portions of a sample from the exit of the thermal converter stage of the amide stage of the process. Water and methanol were then added, and the mix was taken to esterification reaction temperature. Crude MMA was then distilled off from the mix, and this was analysed by GC and GC coupled with mass spectroscopy to look for the presence of any new trace impurities. The results are shown in Table 4, in which it can be seen that both candidates contained no trace materials.

TABLE 4
Product Quality Experiments

| | Trace Impurities Found By GC - Mass |
|---|---|
| Candidate Material | Spectroscopic Analysis |
| Control experiment, no | Trace Impurities typical of ACH route |
| surfactant present | process, e.g. methanol, acetone, |
| | dimethyl ether, methacrylonitrile, |
| | methyl propionate, ethyl methacrylate, |
| | methylhydroxyisobuyrate |
| ‘N’ - ethoxylated, ‘N’ - tallow | As control |
| substituted propylene diamine | |
| (av 10 EO units per molecule) | |
| Branched C13 alkyl | As control |
| ethoxylate/propoxylate | |
| (av 20 to 30 PO/EO units per | |
| alcohol, of which 90% PO) | |

### Example 6: Minimum Effective Level

Two of the candidate surfactant materials were subjected to a further test to determine the lowest concentration that could be used while still observing a discernible effect on the polymer tar in the esterification stage. Further model esterification reactions were carried out by the lab scale batch esterification method outlined in the section above, except that iml of a solution containing 5% of polymer tar in concentrated sulphuric acid solvent had been added as a means of exaggerating the effect of the surfactant and making it easier to see. In the acetone cyanohydrin process for the continuous manufacture of MAA or MMA it is typical to express the concentrations or levels of other raw materials or additives to the process as a fraction of the feed-rate of the main raw material, the Acetone Cyanohydrin. In the present case the concentration of surfactants in the process was expressed in terms of parts per million “ppm” based on the ACH feed-rate. A series of batch esterification reactions was carried out at lab scale where the concentration of the surfactant in the first reaction was set at 5000 ppm. Subsequent reactions were done at gradually reducing levels of surfactant until the level at which there was no longer a discernible effect of polymer tar dispersal had been identified. The level was then increased to 2× this value, and second, confirmatory experiments carried out.

TABLE 5
Minimum Effective Level

| | Minimum Effective Level |
|---|---|
| Candidate Material | (ppm, based on ACH) |
| ‘N’ - ethoxylated, ‘N’ - tallow | 250 |
| substituted propylene diamine | |
| (av 10 EO units per molecule) | |
| Branched C13 alkyl | 750 |
| ethoxylate/propoxylate | |
| (av 20 to 30 PO/EO units per | |
| alcohol, of which 90% PO) | |

### Example 7: Extension of Time Between Stoppages Caused by Polymer Tar Blockage at Production Scale

The candidate surfactant with the lowest minimum effective level was tested at production scale, by carrying out a trial with continuous addition for the complete period between stoppages for clean down. The trial was carried out on a continuous production plant, which was designed to operate at ACH feed-rates of up to 13 te/hr.

Those skilled in the art will recognise that on such plants there are many factors which can affect the rate at which polymer tar is produced, and also the number of blockages which are caused by accumulation of polymer tar. Factors that affect the number of stoppages caused by polymer tar include:

Average Plant Rate: The lower the average plant rate is, the longer the residence time in the amide stage of the process vessels becomes, and the more tar is produced

Number of Plant Hold Periods: Stopping and holding up of process material causes more tar generation because of the effect this has on extending the normal residence times of the material in the vessels, and also allows opportunities for accumulation and blockage due to disengagement of the tar, which is less dense and tends to float on the spent acid in the esterifiers.

Quality of the ACH raw material: Poor quality ACH has been shown to give rise to a greater level of generation of polymer tar

Levels and types of polymerisation inhibitors: This is particularly important in the amide stage of the process, where the majority of the components of polymer tar are formed

Levels of solvent-like components remaining in the spent acid at the end of the stripping stage: It is broadly recognised that the levels and types of solvent-like components in the spent acid, such as Methanol, Acetone, Methacrylic acid, Methylmethacrylate and Hydroxyisobutyric acid, have an effect on the nature of the polymer tar that is found in the esterification vessels. Higher levels of these components lead to polymer tar which is less viscous and sticky, with a lower tendency to accumulate and cause blockages.

The continuous production trial of the preferred candidate surfactant material was designed to take into account two periods of operation at similar production rates, with no surfactant addition. These periods were considered control periods for comparison purposes.

The period between stoppages for clean down, and the mass of polymer tar removed at the shut-down were used as indicators of the performance of the surfactant. The results of the trial are shown in Tables 6 and 7 below, in which it can be seen that a significant lengthening of the period between enforced stoppages, and a reduction in the mass of polymer tar removed are both evident, despite those factors which are known to cause accumulation of polymer tar being worse in the trial period compared with the two control periods.

TABLE 6
Conditions for Continuous Production Trial

| | Control Period | Control Period | Surfactant |
|---|---|---|---|
| Condition | 1 | 2 | Trial Period |
| Average | 10.0 | 9.1 | 8.7 |
| Production rate | | | |
| (ACH feed-rate, | | | |
| te/hr) | | | |
| Number and | Average of 2 | As control | As control |
| duration of | per week, short | period 1 | period 1 |
| Stoppages | duration | | |
| | associated with | | |
| | plant trips | | |
| ACH Quality | Average 0.8% | As for control | As for control |
| (represented by | acetone | period 1, ACH | period 1, ACH |
| the major impurity | | from same | from same |
| % w/w acetone) | | stock used | stock used |
| Process | 300 ppm | As for control | As for control |
| Inhibition, amide | Phenothiazine, | period 1 ACH | period 1 ACH |
| stage (Type and | dissolved in ACH | from same | from same |
| level, expressed as | prior to feed into | stock used | stock used |
| ppm of ACH | amide stage | | |
| feed-rate) | | | |
| Levels of | Average 0.45% | As for control | As for control |
| solvent-like | MAA in spent | period 1 | period 1 |
| components in | acid exit | | |
| spent acid | esterification | | |
| (represented | | | |
| by % MAA) | | | |

TABLE 7
Results from Continuous Production Trial

| | Control Period | Control Period | Surfactant |
|---|---|---|---|
| Success Criteria | 1 | 2 | Trial Period |
| Period | 30 | 20 | 38 |
| between enforced | | | |
| stoppages for clean- | | | |
| out of polymer tar | | | |
| (days) | | | |
| Mass of | 10 | 12 | 6 |
| polymer tar removed | | | |
| during clean down | | | |
| after period of | | | |
| operation (te) | | | |

1. A method of preventing polymer tar build-up in ACH production of methyl methacrylate (MMA) and/or methacrylic acid (MAA) in which one or more surfactants are contacted with a hydrolysis and optional esterification stage reaction medium, the one or more surfactants being selected from:—
a) C₁₀ to C₃₀ alcohol ethoxylates with an average of 5 to 100 ethylene oxide units per molecule;
b) alkyl, hydrogen, —O—[CH₂CH₂O]ₓH and/or —O—[CH₂CH₂CH₂O]_{x′}H N-substituted alkylene di- or triamines with an average of 1 to 30 total ethylene oxide and propylene oxide repeating units per molecule and wherein x and x′ are from 1 to 30; and
c) C₁₀ to C₃₀ alcohol ethoxylate, propoxylates with an average of 5 to 100 total propylene oxide and ethylene oxide units per molecule, which units are in a random, block or alternating sequence or a combination thereof.

2. The method according to claim 1 wherein the surfactants are defined as:—
a) C₁₀ to C₃₀ alcohol ethoxylates with an average of 5 to 100 ethylene oxide units per molecule represented as formula I
R′″—O—[CH₂CH₂O]ₙ—H I
wherein R′″ is a C₁₀ to C₃₀ linear or branched alkyl group, and n is on average 5-100,
b) alkyl, hydrogen, —O—[CH₂CH₂O]_{X}H and/or —O—[CH₂CH₂CH₂O]_{X′}H N-substituted alkylene di- or triamines represented by formula II 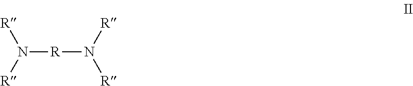
wherein 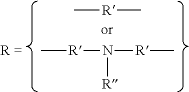
wherein R′ is a —C₂H₄— (ethylene) or C₃H₆— (propylene) group, each R″ is alkyl, hydrogen, —O—[CH₂CH₂O]ᵥH or —O—[CH₂CH₂CH₂O]_{v′}H, wherein at least one R″ group, but not more than two R″ groups, is alkyl, preferably branched or linear C₁₀ to C₃₀ alkyl, and wherein at least one R″ group is —O—[CH₂CH₂O]ᵥH or —O—[CH₂CH₂CH₂O]_{v′}H, and wherein the total average number of —[CH₂CH₂O]— and —[CH₂CH₂CH₂O]— repeating units per molecule of alcohol is from 1 to 20, and wherein v and v′ are from 1 to 20 with the proviso that the total average number of such units does not exceed 20; and/or
c) C₁₀ to C₃₀ alcohol ethoxylate, propoxylate is represented by general formula III
R′″″-O—[CH₂CH₂O]ₙ—[CH₂CH₂CH₂O]ₘ—H III
where R′″″ is a linear or branched C₁₀ to C₃₀ alkyl group and n+m is on average 5-100 per molecule, wherein the —CH₂CH₂O— and —CH₂CH₂CH₂O— units are in a random, block or alternating sequence or a combination thereof.

3. The method according to claim 1, wherein the substituted alkylene di- or triamine b) is of formula IIa 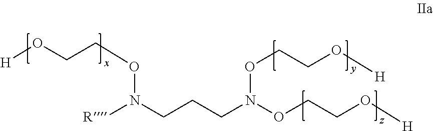
wherein R″″ is derived from tallow fatty acid such as tallow alkyl and x+y+z is on average 5-15 per molecule.

4. The method according to claim 1, wherein the surfactant c) has an average of 20 to 50 total ethylene and propylene oxide units per alcohol and the alcohol group is a C₁₃ to C₁₅ alcohol represented as C₁₃ to C₁₅ alkoxy group.

5. The method according to claim 1, wherein in surfactant c), 1-99% of the units are propylene oxide units.

6. The method according to claim 2, wherein in surfactant c), n:m is between 1:4 to 1:19.

7. A method of producing methyl methacrylate (MMA) or methacrylic acid (MAA) comprising the steps of:—
converting acetone cyanohydrin to methacrylamide (MAM) using concentrated sulphuric acid and thermal treatment;
hydrolysing the MAM to MAA; and
optionally, esterifying MAA to MMA using methanol, wherein one or more surfactants are contacted with a hydrolysis and optional esterification stage reaction medium, the one or more surfactants being selected from:
a) C₁₀ to C₃₀ alcohol ethoxylates with an average of 5 to 100 ethylene oxide units per molecule;
b) alkyl, hydrogen, —O—[CH₂CH₂O]_{X}H and/or —O—[CH₂CH₂CH₂O]_{X′}H N-substituted alkylene di- or triamines with an average of 1 to 30 total ethylene oxide and propylene oxide repeating units per molecule and wherein x and x′ are from 1 to 30; and
c) C₁₀ to C₃₀ alcohol ethoxylate, propoxylates with an average of 5 to 100 total propylene oxide and ethylene oxide units per molecule, which units are in a random, block or alternating sequence or are a combination thereof.

8. The method according to claim 7, wherein the hydrolysis or esterification stage reaction medium comprises a concentrated sulphuric acid solution containing MAM, streams of fresh water, optionally methanol and/or optionally recycle streams such as waste water or streams from refining stages of the process.

9. The method according to claim 7, wherein the one or more surfactants are added to the process in liquid form.

10. The method of claim 7, wherein addition of the one or more surfactant within a hydrolysis or esterification medium vessel is achieved by separate addition to the reaction medium, optionally, at the same time as one or more of the other process streams or a via a mixer placed in one of the other incoming process streams such as an in-line static mixer.

11. The method of according to claim 10, wherein a suitable other incoming stream is the fresh water and/or methanol stream or a recycle stream from the refining stage.

12. The method according to claim 2, wherein the substituted alkylene di- or triamine b) is of formula IIa 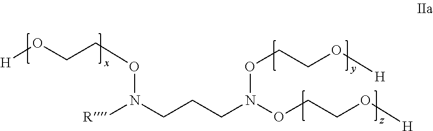
wherein R″″ is derived from tallow fatty acid such as tallow alkyl and x+y+z is on average 5-15 per molecule.

13. The method according to claim 2, wherein the surfactant c) has an average of 20 to 50 total ethylene and propylene oxide units per alcohol and the alcohol group is a C₁₃ to C₁₅ alcohol represented as C₁₃ to C₁₅ alkoxy group.

14. The method according to claim 2, wherein in surfactant c), 1-99% of the units are propylene oxide units.

15. The method according to claim 4, wherein in surfactant c), 1-99% of the units are propylene oxide units.

16. The method according to claim 13, wherein in surfactant c), n:m is between 1:4 to 1:19.

17. The method according to claim 14, wherein in surfactant c), n:m is between 1:4 to 1:19.

18. The method according to claim 9, wherein the liquid is either a substantially pure liquid at the temperature of addition or in the form of a solution.
